(19) United States
(12) Patent Application Publication
(10) Pub. No.: US 2009/0171171 A1
(43) Pub. Date: Jul. 2, 2009

(54) OXIMETRY SENSOR OVERMOLDING LOCATION FEATURES

(75) Inventors: George L. Matlock, Pleasanton, CA (US); Joshua Singh, Longmont, CO (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/340,961

(22) Filed: Dec. 22, 2008

Related U.S. Application Data
(60) Provisional application No. 61/009,710, filed on Dec. 31, 2007.

(51) Int. Cl. A61B 5/1455

(52) U.S. Cl. 600/310

(57) ABSTRACT

The present disclosure relates to a frame for a sensor. In an embodiment, the frame includes a frame body and one or more surface features configured to position the frame body within a mold. In certain embodiments, some or all of the frame may be overmolded such that the overmold material is substantially flush with terminal surfaces of the respective one or more surface features.

### RELATED APPLICATION

This application claims priority from U.S. Provisional Application No. 61/009,710, filed, Dec. 31, 2007, which is hereby incorporated by reference herein in its entirety.

### BACKGROUND

The present disclosure relates generally to medical devices and, more particularly, to sensors used for sensing physiological parameters of a patient.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring physiological characteristics. Such devices provide doctors and other healthcare personnel with the information they need to provide the best possible healthcare for their patients. As a result, such monitoring devices have become an indispensable part of modem medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Pulse oximeters typically utilize a non-invasive sensor that is placed on or against a patient's tissue that is well perfused with blood, such as a patient's finger, toe, forehead or earlobe. The pulse oximeter sensor emits light and photoelectrically senses the absorption and/or scattering of the light after passage through the perfused tissue. The data collected by the sensor may then be used to calculate one or more of the above physiological characteristics based upon the absorption or scattering of the light. More specifically, the emitted light is typically selected to be of one or more wavelengths that are absorbed or scattered in an amount related to the presence of oxygenated versus de-oxygenated hemoglobin in the blood. The amount of light absorbed and/or scattered may then be used to estimate the amount of the oxygen in the tissue using various algorithms.

In many instances, it may be desirable to employ, for cost and/or convenience, a pulse oximeter sensor that is reusable. Such reusable sensors, however, may accumulate detritus and other biological or non-biological debris after repeated use. In addition, such reusable sensors may be difficult to sanitize. Furthermore, such sensors may be uncomfortable for some patients due to the difficulty of sizing the sensors to accommodate a range of finger sizes.

### SUMMARY

Certain aspects commensurate in scope with the disclosure are set forth below. It should be understood that these aspects are presented merely to provide the reader with a brief summary of certain embodiments and that these aspects are not intended to limit the scope of the disclosure.

In accordance with one aspect of the present disclosure, there is provided a sensor frame. The sensor frame comprises a frame body. In addition, the sensor frame comprises at least one surface feature configured to position the frame body within a mold.

In accordance with another embodiment, there is provided a sensor assembly. The sensor assembly comprises a light emitting component, a photodetecting component, and a flex circuit connecting at least the light emitting component and the light detecting component. In addition, the sensor assembly comprises a frame having one or more surface features. The sensor assembly also comprises an overmold material disposed over at least part of the frame such that the surface of the overmold material is substantially flush with terminal surfaces of the respective one or more surface features.

In accordance with a further embodiment, a method for manufacturing a sensor is provided. The method includes the act of positioning a frame within a mold such that surface features on at least part of the frame restrict the motion of the frame within the mold. The frame is coated in the mold.

### BRIEF DESCRIPTION OF THE DRAWINGS

Advantages of the disclosure may become apparent upon reading the following detailed description and upon reference to the drawings in which:

FIG. 1 illustrates a patient monitoring system coupled to a multi-parameter patient monitor and a sensor, in accordance with an embodiment;

FIG. 2 illustrates a perspective view of an internal frame for use in a patient sensor, in accordance with an embodiment; and

FIG. 3 illustrates a cross section view of an overmolded patient sensor, in accordance with an embodiment.

### DETAILED DESCRIPTION

One or more embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

To address certain problems associated with the use of reusable sensors, it may be desirable to provide an overmolding on a sensor to facilitate cleaning of the sensor as well as to provide greater patient comfort. With this in mind, it may be desirable to provide a method and an apparatus for securing and positioning a frame of such a sensor in a mold during the overmolding process. Embodiments may include position pins or features disposed on the frame of the sensor to position the frame within a mold. The pins or other positioning features may be configured to orient and secure the frame within the mold. In an embodiment, the pins or features may further determine the thickness of the overmolding layer cast on the frame. After the frame is overmolded, the pins may be flush with the overmolding surface and, thus, do not leave holes, pockets and/or other breaks in the overmolding that might otherwise be present. Accordingly, eliminating such holes and pockets may prevent accumulation of biological debris and/or fluids on the pulse oximetry sensor, enabling its simple cleaning and better facilitating its prolonged reusability.

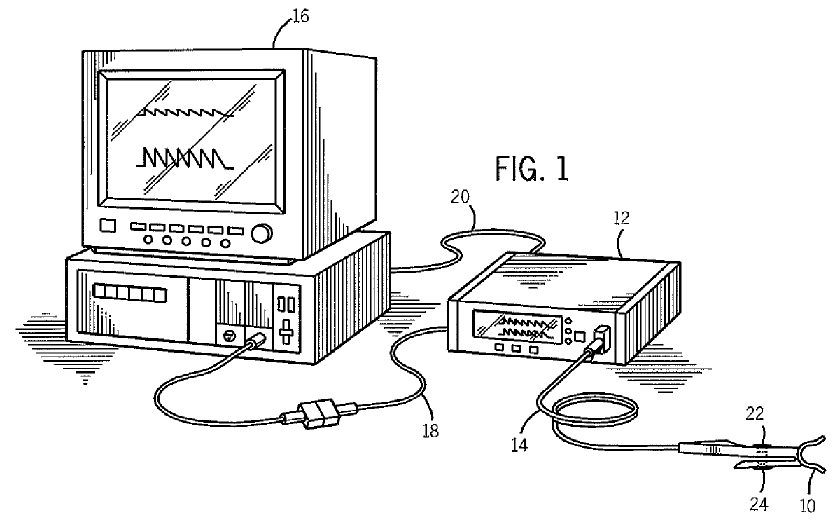

Prior to discussing examples of such sensors in detail, it should be appreciated that such sensors are typically designed for use with a patient monitoring system. For example, referring now to FIG. 1, a sensor 10 according to the present disclosure may be used in conjunction with a patient monitor 12, in an embodiment. In the depicted embodiment, a cable 14 connects the sensor 10 to the patient monitor 12. As will be appreciated by those of ordinary skill in the art, the sensor 10 and/or the cable 14 may include or incorporate one or more integrated circuit devices or electrical devices, such as a memory, processor chip, or resistor, that may facilitate or enhance communication between the sensor 10 and the patient monitor 12. Likewise the cable 14 may be an adaptor cable, with or without an integrated circuit or electrical device, for facilitating communication between the sensor 10 and various types of monitors, including older or newer versions of the patient monitor 12 or other physiological monitors. In some embodiments, the sensor 10 and the patient monitor 12 may communicate via wireless means, such as using radio, infrared, or optical signals.

In such embodiments, a transmission device (not shown) may be connected to the sensor 10 to facilitate wireless transmission between the sensor 10 and the patient monitor 12. As will be appreciated by those of ordinary skill in the art, the cable 14 (or corresponding wireless transmissions) may be used to transmit control or timing signals from the monitor 12 to the sensor 10 and/or to transmit acquired data from the sensor 10 to the monitor 12. In some embodiments, however, the cable 14 may be an optical fiber that allows optical signals to be conducted between the monitor 12 and the sensor 10.

In an embodiment, the patient monitor 12 may be a suitable pulse oximeter, such as those available from Nellcor Puritan Bennett LLC. In an embodiment, the patient monitor 12 may be a monitor suitable for measuring tissue water fractions, or other body fluid related metrics, using spectrophotometric or other techniques. Furthermore, the monitor 12 may be a multi-purpose monitor suitable for performing pulse oximetry and measurement of tissue water fraction, or other combinations of physiological and/or biochemical monitoring processes, using data acquired via the sensor 10. Furthermore, to upgrade conventional monitoring functions provided by the monitor 12 to provide additional functions, the patient monitor 12 may be coupled to a multi-parameter patient monitor 16 via a cable 18 connected to a sensor input port and/or via a cable 20 connected to a digital communication port.

The sensor 10, in the embodiment depicted in FIG. 1, is a clip-style sensor that is overmolded to provide a unitary or enclosed assembly. In an embodiment, the sensor 10 may include an emitter 22 and a detector 24 which may be of any suitable type. For example, the emitter 22 may be one or more light emitting diodes adapted to transmit one or more wavelengths of light, such as in the red to infrared range, and the detector 24 may be a photodetector, such as a silicon photodiode package, selected to receive light in the range emitted from the emitter 22. In the depicted embodiment, the sensor 10 is coupled to a cable 14 that is responsible for transmitting electrical and/or optical signals to and from the emitter 22 and detector 24 of the sensor 10. The cable 14 may be permanently coupled to the sensor 10, or it may be removably coupled to the sensor 10—the latter alternative being more useful and cost efficient in situations where the sensor 10 is disposable.

For pulse oximetry applications using either transmission or reflectance type sensors, the oxygen saturation of the patient's arterial blood may be determined using two or more wavelengths of light, most commonly red and near infrared wavelengths. Similarly, in other applications, a tissue water fraction (or other body fluid related metric) or a concentration of one or more biochemical components in an aqueous environment may be measured using two or more wavelengths of light, most commonly near infrared wavelengths between about 1,000 nm to about 2,500 nm. It should be understood that, as used herein, the term “light” may refer to one or more of infrared, visible, ultraviolet, or even X-ray electromagnetic radiation, and may also include any wavelength within the infrared, v parts that are separately formed. In such embodiments, the different parts may be formed from the same or different materials. For example, in implementations where different parts are formed from different materials, each part may be constructed from a material having suitable mechanical and/or chemical properties for that part. The different parts may then be joined or fitted together to form the internal frame 40. Alternatively, the internal frame 40 may be formed as multiple parts that are joined together to form the internal frame 40.

In the depicted embodiment, the skeletal frame 40 of the pulse oximetry sensor 10 includes location pins 44-50 disposed on the bottom structural support 41. In one embodiment, the location pins 44-50 are tubular structures extending outward from the structural support 41. During overmolding of the skeletal frame 40, the location pins 44-50 may be properly position and secure the skeletal frame 40 within an overmolding fabrication tool, such as a mold suitable for injection molding. In other words, it may be desirable to overmold various portions of the skeletal frame 40 in conformity with certain design and/or operational specifications, such as overmolding thickness, surface morphology, overmolding repeatability and so forth. In one example of such a process, the internal frame 40 may be positioned within a die or mold of the desired shape for the sensor 10 such that the location pins 44-50, or other similar location protrusions or features, secure the frame 40 within a mold or die so that the walls of the mold or die are the desired distance from each part of the frame 40. Accordingly, the location pins 44-50 (or other location features on the frame 40) generally ensure that the frame 40 remains properly oriented within the fabrication tool during the fabrication process. A molten or otherwise unset overmold material may then be injected into the die or mold where it coats the frame 40 with the desired thickness of overmold material at each location.

In an embodiment, a molten thermoplastic elastomer at between about 400° F. to about 450° F. is injected into the mold. The overmold material may then be set, such as by cooling for one or more minutes or by chemical treatment, to form the sensor body about the internal frame 40. Such an overmolding process may result in thermal expansions and subsequent contractions of the frame and the surrounding overmolding material, resulting in movement and/or misalignment of the frame 40 relative the enveloping overmolding material. Accordingly, the location pins 44-50 may be configured to mitigate or prevent such dislocations of the frame 40 by securely holding the frame 40 against the fabrication tool during the overmolding process.

In certain embodiments, the locations pins 44-50 may make unnecessary other holding and/or retaining mechanisms that would otherwise be used to secure the frame 40 within a mold or die. For example, removable pins or assemblies that might otherwise be used to secure the frame 40 within the mold or die are unnecessary because the location features are instead provided on the frame itself. This allows the sensor 10 to be manufactured without the holes and so forth that would otherwise be present on the sensor 10 upon removal of such pins or devices after the overmolding process was completed. Further, the length of the location pins 44-50 may, in part, determine that thickness of the overmold layer disposed on the skeletal frame 40. In this manner, the overmolded skeletal frame 40 may yield a spectrophotometric sensor, such as the sensor 10, having a smooth continuous surface which may be easily cleaned and reused repeatedly.

Other examples of mold or die location features are also possible. For example, in an embodiment, the depicted skeletal frame 40 also includes location tabs 52 and 54 disposed on the lower structural support 41. The location tabs 52 and 54 may, too, be adapted to securely hold and spatially orient the skeletal frame 40 during the overmolding fabrication process. In other embodiments, other tabs, pins and/or notches may be used to maintain the skeletal frame 40 while overmolding material is cast about the skeletal frame 40. Similarly, prongs 56-62 disposed at one end of the bottom support 41 may be utilized as positioning markers for aligning the frame 40 appropriately in a fabrication tool during the overmolding process of the frame 40. For example, the prongs 56-62 may maintain the frame 40 in place so that during high volume manufacturing, repeatability of desirable overmolding features can be achieved.

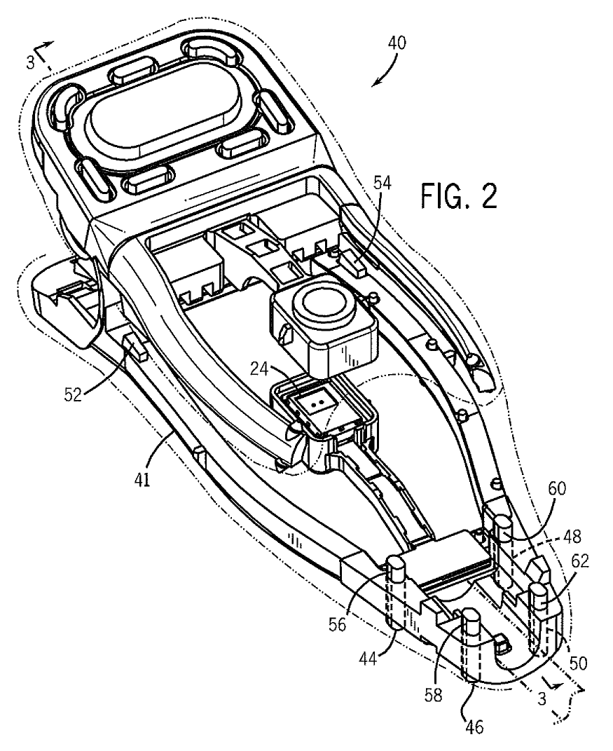

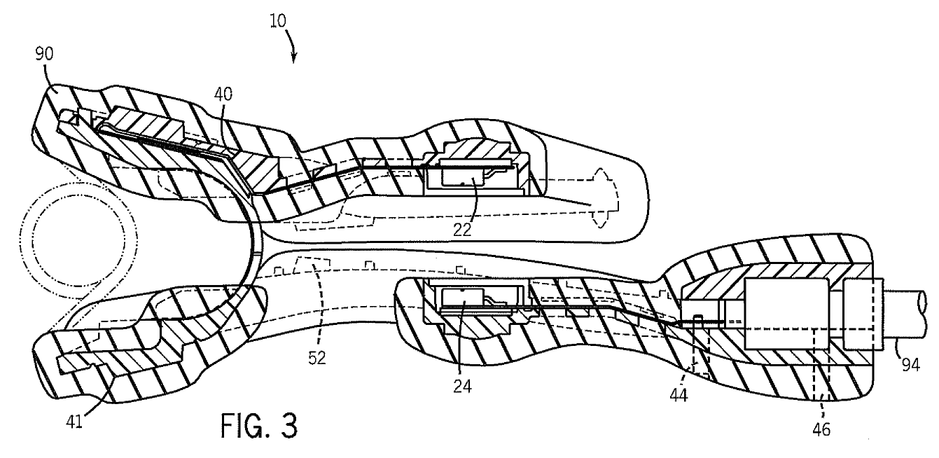

Turning now to FIG. 3, a cross-sectional view of the sensor 10 taken along line 3 of FIG. 2 is depicted in accordance with an embodiment, FIG. 3 depicts the sensor 10 coated with overmolding 90 covering the skeletal frame 40. The overmolding 90 of the sensor 10 may be formed by various molding processes, such as those described above. The frame 40 may be coated to form a unitary or integral sensor assembly, as depicted in FIGS. 3. Such an overmolded embodiment may result in a sensor assembly in which the internal frame 40 is completely or substantially coated.

In the embodiment depicted in FIG. 3, once the skeletal frame 40 is overmolded, the external surfaces of the location pins 44 and 46 may become enveloped by the overmold material except at their end surface, such that the end surface of the location pins 44-50 is generally flush with the surface of the overmolding 90. Such an overmolded sensor assembly may be generally or substantially free of crevices, gaps, junctions or other surface irregularities typically associated with a multi-part construction which may normally allow the accumulation of biological detritus or residue. Accordingly, in embodiments in which the coating 90 employed is liquid or fluid tight, such a sensor 10 may be easily maintained, cleaned, and/or disinfected by immersing the sensor into a disinfectant or cleaning solution or by rinsing the sensor 10 off, such as under running water.

FIG. 3 further depicts a cable 94 connecting a flex circuit of the sensor 10 to an external power source providing electrical current powering the flex circuit. In an embodiment, the cable 94 may join the flex circuit at contact points disposed within the sensor 10. In the illustrated embodiment, the cable 94 is partially coated with the overmolding 90 so that it is securely affixed to the sensor 10.

While the medical sensors 10 discussed herein are some examples of overmolded or coated medical devices, other such devices are also contemplated and fall within the scope of the present disclosure. For example, other medical sensors and/or contacts applied externally to a patient may be advantageously applied using an overmolded sensor body as discussed herein. Examples of such sensors or contacts may include glucose monitors or other sensors or contacts that are generally held adjacent to the skin of a patient such that a conformable and comfortable fit is desired. Similarly, and as noted above, devices for measuring tissue water fraction or other body fluid related metrics may utilize a sensor as described herein. Likewise, other spectrophotometric applications where a probe is attached to a patient may utilize a sensor as described herein.

While the disclosure may be suitable to various modifications and alternative forms, embodiments have been shown by way of example in the drawings and have been described in detail herein. However, it should be understood that the disclosure is not intended to be limited to the particular forms disclosed. Rather, the disclosure is intended to encompass all modifications, equivalents, and alternatives falling within the spirit and scope of this disclosure as defined by the following appended claims.

What is claimed is:

1. A sensor frame, comprising:
a frame body; and
at least one surface feature capable of generally positioning the frame body within a mold.

2. The sensor frame of claim 1, wherein the at least one surface feature contacts the mold when the frame body is positioned generally in the mold.

3. The sensor frame of claim 2, wherein the at least one surface feature comprises a pin or tab projecting generally outward from the frame body.

4. The sensor frame of claim 1, wherein the at least one surface feature generally defines an outer boundary of an overmold applied over the frame body.

5. The sensor frame of claim 1, further comprising an overmold material disposed over at least a portion of the frame body.

6. The sensor frame of claim 1, wherein the frame body comprises a polymeric material.

7. A sensor assembly, comprising:
a light emitting component;
a photodetecting component;
a flex circuit operably coupled to the light emitting component and the light detecting component;
a frame comprising one or more surface features, the frame being operably coupled to the flex circuit; and
an overmold material disposed over at least part of the frame such that the surface of the overmold material is generally flush with terminal surfaces of the respective one or more surface features.

8. The sensor assembly of claim 7, wherein the overmold material disposed over the frame comprises a generally smooth surface.

9. The sensor assembly of claim 7, wherein the one or more surface features defines, at least in part, the thickness of the overmold material.

10. The sensor assembly of claim 7, wherein the light emitting component comprises one or more light emitting diodes.

11. The sensor assembly of claim 7, wherein the photodetecting component comprises a photodiode.

12. The sensor assembly of claim 7, wherein the one or more surface features comprise pins or tabs projecting generally outward from the frame.

13. The sensor assembly of claim 7, wherein the one or more surface features are capable of orienting the frame relative to a mold.

14. The sensor assembly of claim 7, wherein the frame comprises a polymeric material.

15. The sensor assembly of claim 7, wherein the overmold comprises a thermoplastic elastomer.

16. A method for manufacturing a sensor, comprising:
positioning a frame generally within a mold such that surface features on at least part of the frame generally restrict the motion of the frame within the mold; and
coating the frame in the mold.

17. The method of claim 16, comprising:
placing at least one of a light emitting component or a photodetecting component on the frame; and
electrically connecting a flex circuit to the light emitting component or the photodetecting component.

18. The method of claim 16, wherein coating the frame comprises overmolding the frame using injection molding techniques.

19. The method of claim 16, wherein the surface features project generally outward from the frame.

20. The method of claim 16, wherein coating the frame comprises overmolding the frame such that the overmold material is generally flush with the terminal surfaces of the surface features.
